(19) United States
(12) Patent Application Publication
(10) Pub. No.: US 2020/0203021 A1
(43) Pub. Date: Jun. 25, 2020

(54) VISUALIZATION OF HEALTH QUALITY MEASURES

(71) Applicant: KONINKLIJKE PHILIPS N.V., EINDHOVEN (NL)

(72) Inventors: JAMES H TORRANCE, ALPHARETTA, GA (US); BRYANT MENN, ALPHARETTA, GA (US)

(21) Appl. No.: 16/644,230

(22) PCT Filed: Sep. 5, 2018
(86) PCT No.: PCT/EP2018/073890

§ 371 Date: Mar. 4, 2020

Related U.S. Application Data
(60) Provisional application No. 62/554,630, filed on Sep. 6, 2017.

(51) Int. Cl. G16H 50/80; G16H 50/70; G06F 16/22; G06F 3/0483

(52) U.S. Cl. CPC G16H 50/80; G06F 3/0483; G06F 16/2246; G16H 50/70

(57) ABSTRACT

In one embodiment, a quality measure analysis system that visually presents a quality measure as a tree structure where anomalies inpatient care or processes are indicated to a user at one or more nodes of the tree to trigger additional investigation.

### FIELD OF THE INVENTION

The present invention is generally related to quality measures in healthcare, and more particularly, the analysis of quality measures.

### BACKGROUND OF THE INVENTION

Government reimbursed healthcare, such as Medicare, is rapidly evolving from volume-based payments to value-based payments to ensure that patients receive quality healthcare while incentivizing healthcare professionals to provide efficient yet responsible care. Medicare, which is administered by Centers for Medicare and Medicaid Services or CMS, is a federal program implemented in the United States to provide heath coverage for people 65 years of age or older and/or for people with certain qualified disabilities. Medicare may be grouped into one of four plans, including Parts A (for in-patient hospital coverage), B (for outpatient services and generally, services not covered by Part A), Part C (which includes Medicare Advantage, and provides Medicare benefits through capitated health insurance), and Part D (prescription drug). Focusing in particular on Medicare Advantage and reimbursement models, one driving force to a value-based payment model is quality measures. In general, quality measures comprise tools to help Medicare programs assess various aspects of care, including health care outcomes, patient perceptions, and organizational structure. One particular quality measure for CMS, referred to as a clinical quality measure, comprises measures of processes, experiences and/or outcomes of patient care, observations and/or treatment that relate to one or more quality aims for health care including whether the care is effective, safe, efficient, patient-centered, equitable, and timely. For example, a quality measure can provide information about whether a health care provider, including a hospital, has provided care to their patients that supports a clinical process found to be effective in reducing complications associated with a specific disease or medical condition or associated with being hospitalized. Stated more generally, a clinical quality measure provides a standardized means of measuring and comparing the delivery of care. A clinical quality measure may be comprised of several parts, including an initial patient population, a denominator population, an exclusion population, an exception population, and a numerator population. The initial patient population includes the set of patients (or episodes of care) to be evaluated by the quality measure. The denominator population comprises a subset of the initial patient population. The exclusion population comprises the members of the denominator that should not be considered for inclusion in the numerator. The exception population comprises the members of the denominator that are considered for membership in the numerator, but are rejected, and meet the logic required for the exception criteria. The numerator population comprises a subset of the denominator, and has criteria including the processes or outcomes expected for each patient, procedure, or other unit of measurement defined in the denominator.

Beginning in 2015, CMS began to penalize (“apply a negative payment adjustment” to) health care providers for not satisfactorily reporting data on quality metrics. For those organizations that did satisfy the reporting requirements, they avoided the penalty and were eligible for an incentive reward. CMS has recently established, under MACRA (Medicare Access & CHIP Reauthorization Act), a quality payment program that has two tracks—Advanced Alternative Payment Models or ARMs and Merit Based Incentive Payment (MIPs). MIPs provides for a positive (or negative when there is no compliance) payment adjustment according to a composite score comprising different weighted categories of quality, improvement activity, and advanced care information (and also includes a cost component that as of 2017 does not contribute to the composite score, but will in the future). There are 270 quality measures at the time of this writing, and a provider needs to decide on six of them for reporting compliance, with one of them being an outcome measure (or high priority measure if outcome measure is not applicable). Each of the quality measures are formatted as specifications that include measure type (e.g., process, intermediate outcome, etc.), a brief description, instructions on reporting and eligibility, denominator instructions including eligible case criteria (e.g., diagnosis codes (ICD codes), patient encounter codes (e.g., CPT, HCPCS), and where applicable, exclusions and/or exceptions. As the details of the program are well documented, further description of the program is omitted here for brevity. In effect, providers are incentivized to improve their score through efficiencies and/or improvements realized in treatment, technology, reporting, etc. according to any or a combination of the components or categories from which the composite score for MIPs is derived. However, the means to assess the measures for areas of improvement and/or indications of anomalies in the data have been primarily relegated to brute-force manual methods of manually reviewing and analyzing the scores, measures, and contributing data.

### SUMMARY OF THE INVENTION

One object of the present invention is to develop a quality measure analysis system that circumvents some of the time and resources involved in determining areas for improvement in patient care and/or processes. To better address such concerns, in a first aspect of the invention, a quality measure analysis system is disclosed that visually presents a quality measure as a tree structure where anomalies in patient care or processes are indicated to a user at one or more nodes of the tree to trigger additional investigation. The invention facilitates the analysis of reported measures and enables improvements in the healthcare process, which in the era of government incentives and/or penalties, helps to realize positive adjustments in reimbursements for government-funded healthcare.

In one embodiment, a threshold difference between a patient count at a first or root level and total patient population provides an indication to the user of a suspected anomaly. For instance, if the patient count reveals a total health care provider patient population of fifty-two thousand patients, and only twenty-two of those patients have a diagnosis of diabetes, which is a relatively common condition, a user of the quality measure analysis system can infer from this discrepancy that there is an error somewhere in the healthcare treatment, coding, and/or reporting process. The visualization of the tree structure provides a simple yet effective indication of the anomaly and triggers further investigation leading toward remedial measures.

In an embodiment, wherein at least one of the multiple nodes comprises a Boolean OR or a Boolean AND, the at least one of the multiple nodes comprising an ordered list of items, each of the items comprising a patient count and a record count for a first condition, the items comprising any one or a combination of current procedural terminology code procedure, a value set procedure, an agent procedure, a custom procedure, analytic tests, demographics, tags, industry standard diagnosis codes, value set diagnosis codes, agent diagnosis codes, or custom diagnosis codes. The hierarchical arrangement of the items (in quantity of patient counts) provides a quick visual of the items most prevalent for a given provider, which can be presented to the provider to confirm this is the provider's intent.

In an embodiment, wherein a threshold difference between the record count and the patient count within at least one of the items provides an indication of a suspected anomaly. For instance, a patient count may be at eleven patients, yet a record count of thirty-three may reveal that each patient was recorded three times, or one or more patients were recorded more than once, which may reveal inefficiencies in the data entry process that analysis and subsequent recommendations may remediate. As another example, the visualization of five patient counts and fifteen records provides an indication to the user that there may be a data entry or otherwise procedural inefficiency (e.g., duplication or more of data record entry for one or more patients). The user, equipped with a combination of experience and the context revealed through the visualization, enables the user to determine whether this suspected anomaly merits further investigation. For instance, a healthy person is expected to have one annual wellness visit, whereas someone with a chronic illness is expected to have a schedule of other visits. The visualization short-circuits the brute force approach of sifting through vast amounts of documentation to present a potential for process improvements and/or improved quality in patient care.

In an embodiment, wherein a relative broadness of the top listed item compared to availability of items corresponding to the first condition provides an indication of a suspected anomaly. For instance, the item may be too generic or too specific and missing patients depending on the coding of the users, which may result in a provider missing out on potential points for a given government incentives program. The ready visualization of this anomaly can trigger the user to consult with the provider in finding more narrowly tailored quality measures where points for improvements can be extracted to realize additional revenue or discern areas for improvement. Alternatively, the discovery of a broad coding practice may lead to recommendations for more precise coding practices to enable the provider to report more specific quality measures.

In an embodiment, wherein the tree structure comprises a numerator component and a denominator component, the numerator component and the denominator component corresponding to respective features of the quality measure, the numerator component and the denominator component comprising one or more levels of the multiple nodes. As noted previously, quality measures may have a numerator component that represents a clinical action to be counted as meeting a quality measure's requirement or specification (e.g., patients whom receive a particular service or obtained a particular outcome that is being measured, such as an HbA1c lab). The tree structure visualizes this component of the quality measure along with the denominator component described collectively above, providing readily ascertainable clues to suspected anomalies in the healthcare process for a given provider.

In an embodiment, wherein a threshold difference between the patient count of the root level and a patient count corresponding to at least a node of the numerator provides an indication to the user of a suspected anomaly. For instance, a user can readily observe from the tree structure that, if there are twenty diabetic patients and only two HbA1c labs recorded, there may be a problem in the treatment or coding process, an issue in the data transfer, communication, and/or transformation process, and/or sources (e.g., any system that for which data is aggregated, including but not limited to manual entry, EHR/EMR, payers, and/or lab feeds) may be missing (e.g., not tied into the data registry), and the user can collaborate with the provider using this information to investigate the problem and find a solution to help the provider realize improvements in patient care and/or processes that may result in financial rewards.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1A is a schematic diagram of an example network environment in which an example quality measure analysis system may be used, in accordance with an embodiment of the invention.

FIG. 1B is a flow diagram of an example data flow for an example quality measure analysis system for the network environment of FIG. 1A, in accordance with an embodiment of the invention.

FIGS. 2-3 are screen diagrams of example graphical user interfaces showing example simple Boolean AND-OR tree structures with Boolean ORs having an ordered list of items for an example quality measure analysis system, in accordance with an embodiment of the invention.

FIG. 4 is a schematic diagram that illustrates a portion of a template tree structure with example denominator item types for an example quality measure analysis system, in accordance with an embodiment of the invention.

FIG. 5 is a schematic diagram that illustrates an example template tree structure with example denominator with exclusions and exceptions and numerator item types for an example quality measure analysis system, in accordance with an embodiment of the invention.

FIG. 6 is a schematic diagram that illustrates an example template tree structure with example denominator and numerator item types without exclusions and exceptions for an example quality measure analysis system, in accordance with an embodiment of the invention.

FIG. 7 is a flow diagram that illustrates an example process for building and presenting a tree structure for an example quality measure analysis system, in accordance with an embodiment of the invention.

FIG. 8 is a schematic diagram that illustrates an example computing device used to present a tree structure to a user for an example quality measure analysis system, in accordance with an embodiment of the invention.

FIG. 9 is a flow diagram that illustrates an example quality measure analysis method, in accordance with an embodiment of the invention.

### DETAILED DESCRIPTION OF EMBODIMENTS

Disclosed herein are certain embodiments of a quality measure analysis system that visually presents a quality measure as a tree structure where anomalies in patient care or processes are indicated to a user at one or more nodes of the tree structure to trigger additional investigation. In one embodiment, the quality measure analysis system is used in conjunction with value-based reimbursement programs (e.g., quality payment program of MACRA) administered by the Centers for Medicare and Medicaid Services or CMS under Medicare Advantage health plans, though not necessarily limited to these programs.

Digressing briefly, and as indicated previously, the emphasis on value-based (versus volume based) fee-for-services payments for government-reimbursed healthcare programs has lead to efforts to find improvements in patient care and/or processes to help healthcare providers (herein, simply providers) realize bonuses for such improvements and avoid penalties for non-compliance. Since quality measures play an important role in the scoring that is used to determine the rewards or penalties, providers employ or contract with analysts to comb through their procedures and charts to determine ways to benefit from the incentives offered by CMS and/or avoid penalties. However, these prior methods consume extensive hours and/or resources. In contrast, certain embodiments of a quality measure analysis system aid a user in the analysis of the measures by providing a visualization of the quality measure and patient and record counts at nodes of the tree structure, revealing suspected anomalies that can be further investigated for potential areas of improvement, which in turn can lead to realization of increased bonuses and/or avoidance of penalties in government sponsored incentive programs.

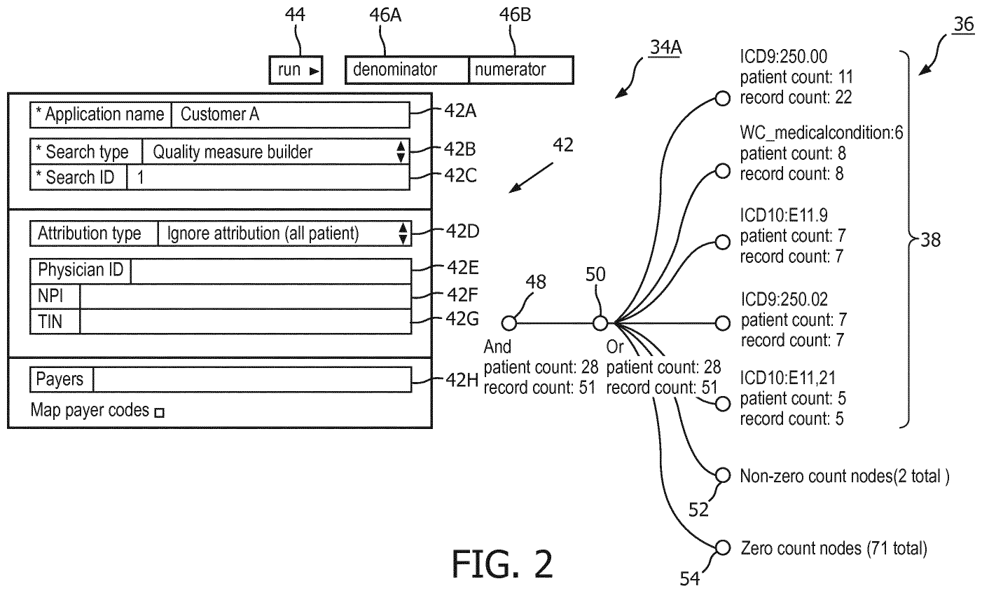

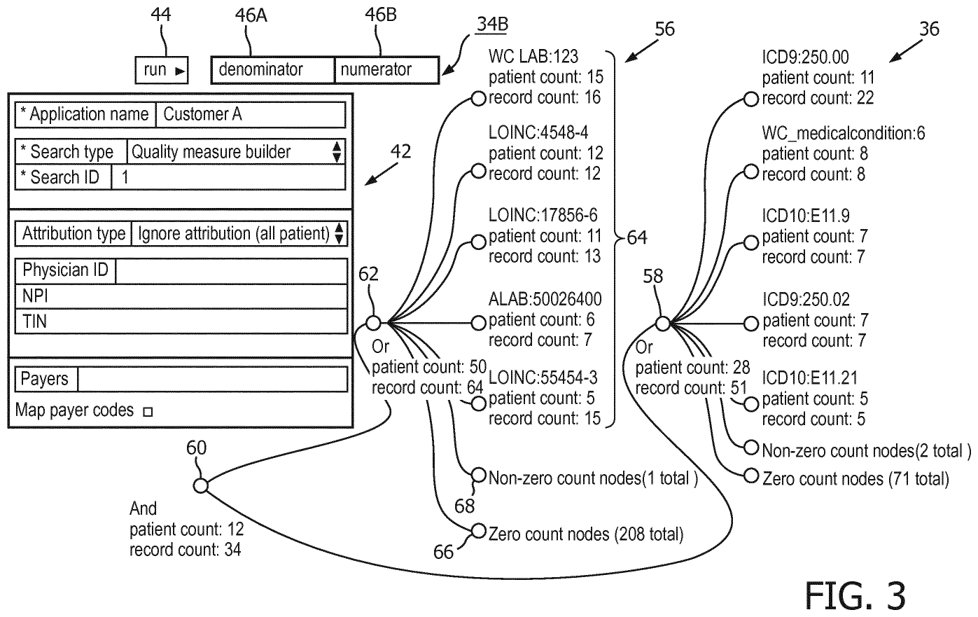

Having summarized various features of certain embodiments of a quality measure analysis system of the present disclosure, reference will now be made in detail to the detailed description of a quality measure analysis system as illustrated in the drawings. While the disclosure is described in connection with these drawings, there is no intent to limit it to the embodiment or embodiments disclosed herein. For instance, though CMS-based quality measures are used in the examples, it should be appreciated by one having ordinary skill in the art in the context of the present disclosure that other quality measures may be used, including those by HEDIS, ACOs (Accountable Care Organizations), provider-configured measures, and/or others whose features can be visualized using a tree structure. Further, though the incentives program of MACRA are referenced in the present disclosure, other incentive programs of the past or in the future for CMS or other organizations of, or affiliated with, government reimbursed healthcare may similarly employ and benefit from certain embodiments of the quality measure analysis system, and hence are contemplated to be within the scope of the disclosure. In addition, some Boolean OR nodes that have been expanded show various types of items, though it should be appreciated by one having ordinary skill in the art in the context of the present disclosure that Boolean AND nodes may be expanded as well. Items comprise cares, medications, labs, and/or vitals, including the same components of submitted quality measures and, in some embodiments, may include other indicators of a health or well-being of an individual, which may include demographic information. Cares refers to procedures, treatment, or generally, encounters between the health care professional and the patient. Medications include prescriptions and/or over-the counter medicines recommended by the health care professional. Labs include procedures that draw blood from or otherwise receive a sample from the patient. Vitals include direct or indirect measures of physiological conditions or parameters of the patient, including heart rate, blood pressure, blood sugar level, etc. In the examples depicted in FIGS. 2-3, for instance, the items may include diagnostic codes (e.g., ICD-9 or -10 codes) and/or various procedure codes, though it should be appreciated by one having ordinary skill in the art in the context of the present disclosure that these are illustrative, non-limiting examples, and that in some embodiments, diagnostic codes other than ICD-9 or -10 codes may be used including SNOMED or any other industry-recognized diagnosis codes, or proprietary codes. In some embodiments, items may comprise any one or a combination of current procedural terminology code procedure, a value set procedure, an agent procedure, a custom procedure, analytic tests, demographics, tags, industry standard diagnosis codes, value set diagnosis codes, agent diagnosis codes, or custom diagnosis codes. Further, although the description identifies or describes specifics of one or more embodiments, such specifics are not necessarily part of every embodiment, nor are all various stated advantages associated with a single embodiment. On the contrary, the intent is to cover all alternatives, modifications and equivalents included within the scope of a quality measure analysis system as defined by the appended claims. For instance, certain embodiments of a quality measure analysis system may be beneficially used for any populations that may be represented as a tree structure. Further, it should be appreciated in the context of the present disclosure that the claims are not necessarily limited to the particular embodiments set out in the description.

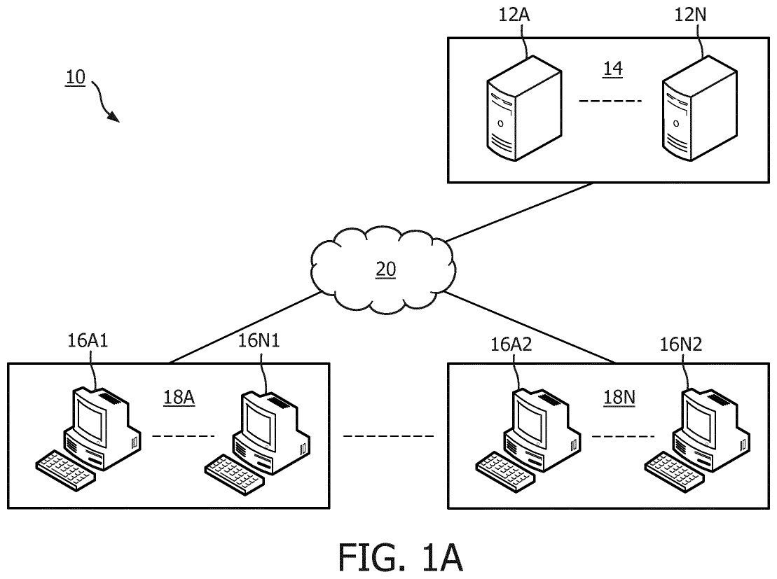

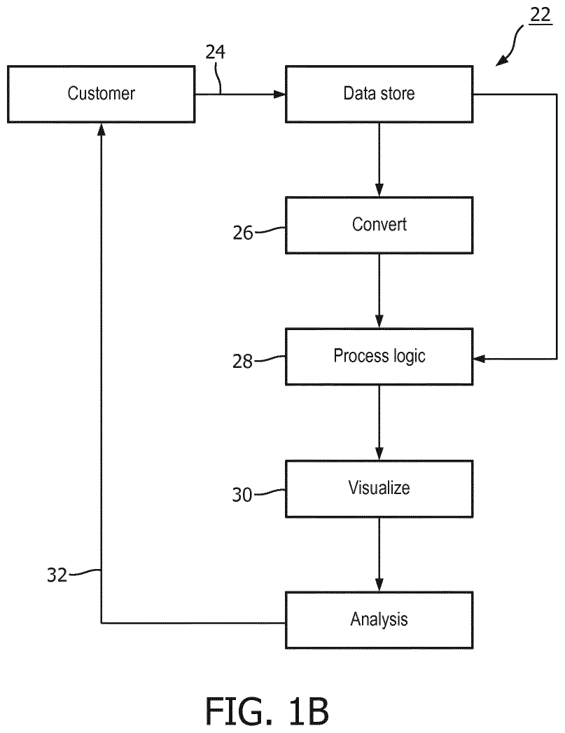

Attention is now directed to FIG. 1A, which is a schematic diagram of an example network environment 10 in which an example quality measure analysis system may be used, in accordance with an embodiment of the invention. It should be appreciated by one having ordinary skill in the art in the context of the present disclosure that the network environment 10 is one example among many, and that some embodiments of a quality measure analysis system may be used in environments with fewer, greater, and/or different components or system architectures than those depicted in FIG. 1A. The network environment 10 comprises a plurality of devices that enable communication of, or access to, information via one or more networks. The depicted network environment 10 comprises one or more server devices 12 (e.g., 12A through 12N) of a server network 14 in communication with client devices 16 (e.g., 16A1 through 16N1, 16A2 through 16N2, etc.) of one or more health services facilities 18 (e.g., 18A through 18N) over one or more networks 20. For instance, the health services facilities 18 may include hospitals and ambulatory care facilities, and users may include physicians, nurse assistants, lab technicians, administrators, and any other users that record patient information via the client devices 16.

The client devices 16 may include desktops, laptops, tablets, among other devices connected directly or indirectly to the network 20. The client devices 16 for each health services facility 18 may be coupled over a local area network (LAN) (e.g., a company intranet), including via wireless LAN (WLAN).

The network 20 may be any type(s) and/or form of network or networks, including a LAN, wide area network (WAN), including the Internet or World Wide Web, a metropolitan area network (MAN), public, private, etc. The network 20 may be comprised of devices interconnected over wireless or wired connections, or a combination of wired and wireless connections. For instance, the network 20 may include any one or combination of the following: a point to point network, a broadcast network, a wide area network, a local area network, a telecommunications network, a data communication network, a computer network, an ATM (Asynchronous Transfer Mode) network, a SONET (Synchronous Optical Network) network, a SDH (Synchronous Digital Hierarchy) network, a wireless network and a wireline network. In some embodiments, the network 20 may comprise a wireless link, such as an infrared channel or satellite band. The topology of the network 20 may be a bus, star, or ring network topology. The network 20 and network topology may be of any such network or network topology as known to those ordinarily skilled in the art capable of supporting the operations described herein.

In one embodiment, the server devices 12 of the server network 14 may provide cloud computing services, including remote data storage, data modeling applications, internet services, security services, content distribution, etc. to the client devices 16 of the health services facilities 18. In some embodiments, the server devices 12 may be coupled via a LAN (or wireless LAN, etc.), or coupled according to geographically separate facilities via the network 20 or other networks, such as to implement plural cloud systems. When embodied as a cloud service or services, the server devices 12 may comprise an internal cloud, an external cloud, a private cloud, or a public cloud (e.g., commercial cloud). For instance, a private cloud may be implemented using a variety of cloud systems including, for example, Eucalyptus Systems, VMWare vSphere®, or Microsoft® HyperV. A public cloud may include, for example, Amazon EC2®, Amazon Web Services®, Terremark®, Savvis®, or GoGrid®. Cloud-computing resources provided by these clouds may include, for example, storage resources (e.g., Storage Area Network (SAN), Network File System (NFS), and Amazon S3®), network resources (e.g., firewall, load-balancer, and proxy server), internal private resources, external private resources, secure public resources, infrastructure-as-a-services (IaaSs), platform-as-a-services (PaaSs), or software-as-a-services (SaaSs). The cloud architecture of the server devices 12 may be embodied according to one of a plurality of different configurations. For instance, if configured according to MICROSOFT AZURE™, roles are provided, which are discrete scalable components built with managed code. Worker roles are for generalized development, and may perform background processing for a web role. Web roles provide a web server and listen and respond for web requests via an HTTP (hypertext transfer protocol) or HTTPS (HTTP secure) endpoint. VM roles are instantiated according to tenant defined configurations (e.g., resources, guest operating system). Operating system and VM updates are managed by the cloud. A web role and a worker role run in a VM role, which is a virtual machine under the control of the tenant. Storage and SQL services are available to be used by the roles. As with other clouds, the hardware and software environment or platform, including scaling, load balancing, etc., are handled by the cloud.

In some embodiments, the server devices 12 may be configured into multiple, logically-grouped servers, referred to as a server farm. The server devices 12 may be geographically dispersed, administered as a single entity, or distributed among a plurality of server farms, executing one or more applications on behalf of one or more of the client devices 16. The server devices 12 within each farm may be heterogeneous. One or more of the server devices 12 may operate according to one type of operating system platform (e.g., WINDOWS NT, manufactured by Microsoft Corp. of Redmond, Wash.), while one or more of the other server devices 12 may operate according to another type of operating system platform (e.g., Unix or Linux). The group of server devices 12 may be logically grouped as a farm that may be interconnected using a wide-area network (WAN) connection or medium-area network (MAN) connection. The server devices 12 may each be referred to as, and operate according to, a file server device, application server device, web server device, proxy server device, or gateway server device.

Referring to FIG. 1B, shown is a flow diagram of an example data flow 22 for an example quality measure analysis system for the network environment 10 of FIG. 1A, in accordance with an embodiment of the invention. It should be appreciated by one having ordinary skill in the art in the context of the present disclosure that the data flow 22 is illustrative of one embodiment, and that in some embodiments, the data flow may be modified (e.g., steps omitted or replaced, added to, etc.). In one embodiment, customers (e.g., providers) communicate patient data and/or procedures (e.g., items, such as components of quality measures, charts, demographics, labs, diagnosis codes, procedures, etc.) to a patient data store (24). In one embodiment, the customers may be associated with the client devices 16 and the patient data store may be a part of the server network 14. The communication of the data may be achieved via the network 20. In one embodiment, the quality measures from the data may be configured in human-readable format. The quality measures are converted (26) to generic, machine-readable format and stored in a definitions store. In one embodiment, one or more persons skilled in one or more quality measure schemes (e.g., clinical quality measures (CQM), PQRS measures, HEDIS, etc.) translates (e.g., using machine tools) the quality measures into a data structure that is used by certain embodiments of a quality measure analysis system to generate the tree structure(s) and corresponding functionality. The translation involves known processes, including taking the natural language of the specification of the quality measures and converting them into computer/machine readable form. In some embodiments, this process may be automated, such as via technology in progress for CMS (e.g., according to the published HL7 CQL specification). A computing device (e.g., processing logic) of the quality measure analysis system receives and processes (28) the patient data from the patient data store and the generic quality measures from a definitions store, and generates a visualization (e.g., the tree structure) (30). The computing device may be a part of the server network 14, or in some embodiments, part of the health services facilities 18, or in some embodiments, comprise functionality distributed over the server network 14 and the health services facilities 18. Based on the results of the visualization-aided analysis of the quality measures, a user (e.g., analyzing the trees) communicates the results to the customer (32). The communication can be achieved via a report communicated via electronic mail, posted to a secured website, or via telephony (orally communicated), among other methods of communicating findings and/or recommendations. In some embodiments, such as where the quality measure analysis system is a client-server application running among client devices 16 and the server devices 12, the analysis may be performed by the customer based on the tree structure rendered at the server device 12 and downloaded or otherwise accessed by the client device 16, which would lead to omission of the communication step (32) for such embodiments.

Referring now to FIG. 2, shown is a screen diagram of an example graphical user interface (GUI) 34A showing an example Boolean AND-OR tree structure 36 that has multiple levels, with Boolean ORs having an ordered list of items 38 for an example quality measure analysis system, in accordance with an embodiment of the invention. It should be appreciated by one having ordinary skill in the art, in the context of the present disclosure, that the GUI 34A is one example illustrative of a simple tree structure 36, and that in practical implementations, the tree structure 36 may be simpler or more extensive depending on the complexity of the quality measure. Further, the formatting of the GUI 34A may be different in some embodiments, but contemplated to be within the scope of the disclosure as long as the tree structure 36 is presented. Though shown with a single Boolean AND node 48 which is the root node in this case at a first hierarchical level, there may be plural AND nodes at subsequent levels corresponding to Boolean AND features of a given quality measure specification. Likewise, though five (5) items 38 are illustrated in FIG. 2, practical implementations may have greater or fewer numbers of Boolean OR items corresponding to given quality measure denominator features. On the left hand side of the GUI 34A are one or more fields (in this example, plural fields) 42 in which a user may enter and/or select information to limit the tree structure to one or more parameters (e.g., to limit patient populations). For instance, the first field 42A enables entry of an application name (e.g., customer, here customer A). A second field 42B enables entry of a search type. The search type may include plural options, including search by saved search, quality measure, or PQRS measure. In other words, searches of data from the patient data store and definitions store is based on the information entered in part in the fields 42. The saved search option is merely a portion of a quality measure or a specific, propriety measure, and provides a single value representing a population. The quality measure search comprises a numerator and denominator with the possibility of exceptions and exclusions. The PQRS option comprises measures specific to PQRS or in some embodiments, MIPs (e.g., PQRS measures are part of MIPs). The search ID field 42C is a database index, which provides an index to a data (e.g., definitions) store. In some embodiments, the index may be replaced with a unique internal identifier. For instance, a user may access the database to look for a specific ID he or she is already familiar with, or the user may log into a customer application, and navigate to the measures desired for analysis, where the measures are part of a uniform resource location (URL) and the ID is the URL. In some embodiments, a scroll-down menu may be used to enter a feature or features of interest (e.g., diabetic condition, measure format according to ACO, year 2016, etc.). In one embodiment, at a minimum, the population of fields 42A-42C is required, whereas the rest of the fields (explained below) may be left blank. Continuing the description of the fields 42, the attribution type 42D provides a mechanism dictating the patient count. The patient count may be further subdivided by physician ID 42E, national provider identifier (NPI), and/or tax identifier (TIN). The user can specify any or all of the fields 42, enabling the user to determine how to associate the physicians to the patients for crafting a database search. In some embodiments, a proprietary association between physician and patient may be entered. For the NPI or TIN based attributions, the user is further enabled to associate a physician to patients via occurrence of a service. The payers field 42H covers insurance carriers, and enables the user to specify for the search, say, only patients that have Medicare/Medicaid, or to limit the search parameters to BlueCross or Aetna patients, as some illustrative examples. Accordingly, through input (e.g., entry of (or selection)) of information in these fields 42, the users are provided options on how they associate physician and patient relationship search criteria.

The GUI 34A further comprises a run button icon 44 that the user may select after inputting the various options in one or more fields 42. The run button icon 44, when selected, prompts the running of the processing portion of the quality measure analysis software to populate and present the tree structure 36 with the desired quality measure items and respective patient and record counts according to the parameters entered in the fields 42. Stated otherwise, input to the fields 42 define what data is extracted from definition stores and patient data stores (described below), and the result of selecting the run button icon 44 is the tree structure 36. The GUI 34A further comprises optional toggle button icons 46, where the visualization can be presented according to the denominator specification 46A or the numerator specification 46B for a given quality measure (e.g., quality measure builder option, PQRS option).

Referring to the tree structure 36, which visualization results from selecting the run button icon 44 (with the denominator button icon 46A selected for the current view), shown in this simple example is a root node 48. The root node 48 is a Boolean AND node at a first level, and represents the entire eligible population for a measure for a particular condition (e.g., in this example, diabetes). In some implementations, the root node may be a Boolean OR node. In some implementations (e.g., extremely simple scenarios), the root node may be a single code (e.g., for some condition, procedure, etc.). In the present example, the root node 48 represents the entire diabetes population for the selected application name and quality measure builder option. The root node 48 has a corresponding patient count of 28 and a record count of 51 in this example. Connected to the root node 48 is a next hierarchical level shown as a Boolean OR node 50. In some implementations, the next level may be a Boolean AND node or a single code (e.g., terminology code). The Boolean OR node is expanded into an ordered list of items 38 corresponding to the condition, each with a respective patient and record count for the eligible population (the diabetic population). In this example, the items 38 include diagnostic codes (e.g., ICD9, ICD10), medical conditions used by the user, and custom codes (e.g., specific to a customer). As indicated above, additional, fewer, and/or other items may be used.

The tree structure 36 visually indicates to the user suspected anomalies associated with this measure and the corresponding eligible population. For instance, referring to the root node 48, it is noted that that there is a patient count of 28 and a record count of 51. Often times a patient item may be submitted multiple times in the healthcare process, which may explain the discrepancy between the numbers. In other words, one suspected anomaly is the disparity between the record and patient count at the root node 48, which may be investigated by the user for inefficiencies in data entry and/or reporting.

Another suspected anomaly immediately evident from the tree structure 36 is for the item 38 labeled ICD9: 250.00. Notably, the patient count is 11, and the record count is 22. One possibility is that each patient was double-coded (or one or more patients had more than two recordings). The user may infer from this discrepancy in record versus patient count that there may be issues in the way data is ingested at the office of the health care provider or in the manner of charting patients (e.g., resulting in multiple records). The user may also infer from the discrepancy that the manner of recording the patient diagnostic codes requires duplication (e.g., a diabetic check-up and an office visit). In other words, though there is a suspected anomaly readily visible from the tree structure 36, it is up to the user to investigate the source of the discrepancy.

The tree structure 36 also provides indications of suspected anomalies from a higher perspective. For instance, observing the root node 48 patient count, and equipped with the knowledge that the provider corresponding to the application name has thousands of patients, the fact that there are only 28 diabetic patients may suggest issues in terms of the scale of numbers (e.g., one expects a much larger diabetic population). The user should investigate such disparities in patient population for the given condition (e.g., diabetes) versus the overall patient database for this customer/provider.

Yet another valuable insight gained from the tree structure 36 is based on the ordered listing of items at the Boolean OR node 50. As indicated previously, the items are sorted, with those items 38 presented in FIG. 2 comprising the top five most used items 38 (e.g., diagnostic or condition codes) for the given quality measure. The top item 38, ICD9:250.00, is actually a very generic code, and yet is the highest reported item 38. The generalness or broadness of the top listed item 38 may present an indication to the user of a suspected anomaly in the sense that the customer may be missing an opportunity for reimbursement due to the generic nature of the top listed code. Alternatively, the discovery of a broad coding practice may lead to recommendations for more precise coding practices (e.g., so the provider(s) can report for more specific measures). Stated simply, an item may be too generic or too specific and may be missing patients depending on coding by the user. The user may be prompted by this anomaly to investigate further with the customer on ways to properly select quality measures to enhance the reimbursement adjustment.

It is noted that the GUI 34A further comprises, in this example, two additional nodes from the Boolean OR node 50, namely, non-zero count node 52 and zero count node 54. The non-zero count node 52 indicates the presence of two child nodes relative to the non-zero node, whereas the zero count node 54 comprises seventy-one (71) child nodes. It is noted that patient and record counts are not presented for the non-zero and zero count nodes 52, 54. The counts indicate additional codes for the given measure, and in the case of the zero count node 54, also reveal that the customer does not have these codes. The nodes 52 and 54 are shown in collapsed state (solid nodes), as compared to the other nodes of this tree structure (open nodes, revealing their expansion to additional nodes). One purpose of the non-zero count node 52 and zero count node 54 is to make the GUI 34 more manageable when a large number of conditions are involved.

Referring to a second example in FIG. 3, shown is a GUI 34B comprising the tree structure 36 corresponding to the denominator and a tree structure 56 corresponding to the numerator based on user selection of the run button icon 44 and selecting the numerator toggle button icon 46B. It is noted that each tree is for a single part or feature of a quality measure (e.g., denominator, numerator, etc.). The fields 42 are as described in association with FIG. 2, and hence discussion of the same is omitted here for brevity. As explained previously, the numerator is a subset of the denominator population (e.g., diabetes population) for whom a process or outcome of care occurs (e.g., Hb1Ac labs). In other words, the tree structure 56 conveys the items for the diabetic denominator population who had, in this example, an Hb1Ac lab corresponding to a given quality measure. Ideally, every diabetic should have at least one Hb1Ac lab administered, since it is a good measure of their health. Accordingly, the numerator and denominator patient count should be 1:1. However, by comparing the patient counts (28) of the denominator at node 58 for the denominator (diabetic population) corresponding to the tree structure 36 with the patient counts (12) at a root node 60 (Boolean AND) of the tree structure 56 corresponding to the numerator, a suspected anomaly is observed—namely, the absence of a 1:1 ratio. Hence, the user is prompted by this indication of the anomaly to investigate further and determine with the customer whether there is an issue in charting or in the process. For instance, based on this suspected anomaly, the user may infer that the customer is not sending the proper data to identify diabetic patients and/or the data is present to identify diabetics, but is missing from the measure builder search construct. It is noted that certain embodiments of the measure quality analysis system may provide an indication to the user of anomalies present on the patient treatment/process side and user and customer side processes. The tree structure 56 is further depicted in this example as having a Boolean OR node 62 connected to the root node 60, the Boolean OR node 62 comprising an ordered list of items 64. Similar to observations with the tree structure 36, suspected anomalies to look for in the ordered list of items 64 are patients with possible multiple chartings. For instance, the item identified as LOINC:55454-3 from the list of items 64 provides an indication of possible multiple chartings, as there are 5 patients yet 15 records. From this anomaly, the user may infer that each patient has 3 charts or the like, suggesting a quality issue worth investigating further. In effect, the anomalies indicated to a user by these tree structures 56 and/or 36 often manifest themselves as the presence of too much data and/or the absence of enough data. Another suspected anomaly is indicated by the presence of the user's own (internal) code for the top-listed item 64-WC_Lab:123. This may occur during the conversion process described in association with FIG. 1B above. An issue with this code is the inability to bill anyone using that code or report it. Possible remedial measures include discussing with a measure steward to find a way to report this code, or equate it to a standard Hb1Ac lab.

Another anomaly that the structured trees 36, 56 bring to the attention of the user is the absence of missing items 64 (e.g., missing diagnosis codes). For instance, if the customer (customer A) associated with the inputted application name uses a custom code for charting ICD codes, and it was stored in the definitions store, one would expect the presence of that code among the items 64 or 68 (or items 38 or 52, FIG. 2). The absence of that custom code among the items 38, 52, 64, 68 may suggest to the user the need to expand the given quality measure to include it, prompting an investigation into the conversion process. Alternatively, the expectation of certain codes from a given, standardized quality measure and the absence of any record of it may present an anomaly that merits further investigation. The zero count node 66 is shown with a total of 208. The non-zero count node shows a total of one. Note that the interpretation of the tree structures 36, 56 and/or the various nodes may be dependent on the knowledge by the user of coding practices of a customer. For instance, if the user knows that the customer coded a particular lab with 10 different types of codes, then this may be cause for concern because only 6 codes are captured in the result. On the other hand, if there are only 2 codes expected for this particular lab, then investigation into how the additional codes arose may be warranted.

It should be appreciated by one having ordinary skill in the art in the context of the present disclosure that these tree structures 36, 56 are illustrative of simple examples, and that in practical applications, there may be more Boolean AND nodes and/or Boolean OR nodes corresponding to different features of a given quality measure specification at one or more levels, or fewer nodes (e.g., a single root node) as well as different patient and/or record counts, among other variations.

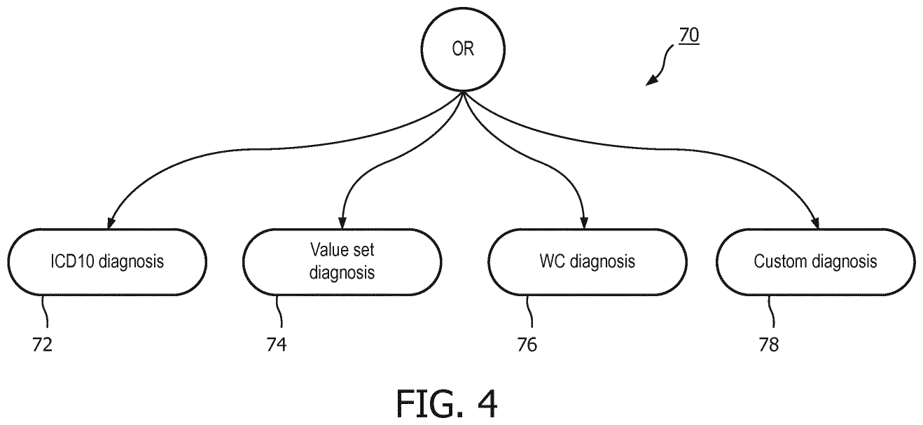

Referring now to FIG. 4, shown is a portion 70 of an example template tree structure that illustrates example denominator item types for an embodiment of an example quality measure analysis system. The item types depicted in this example include ICD10 diagnosis codes, value set diagnosis codes 74, user diagnosis codes 76 (e.g., as generated during the conversion process illustrated in FIG. 1B), and custom diagnosis codes 78 (e.g., tailored for use for a specific customer). Note that the item types listed here are a few examples, and that other and/or additional item types may be used, including procedures, analytic tests, demographics, and/or tags.

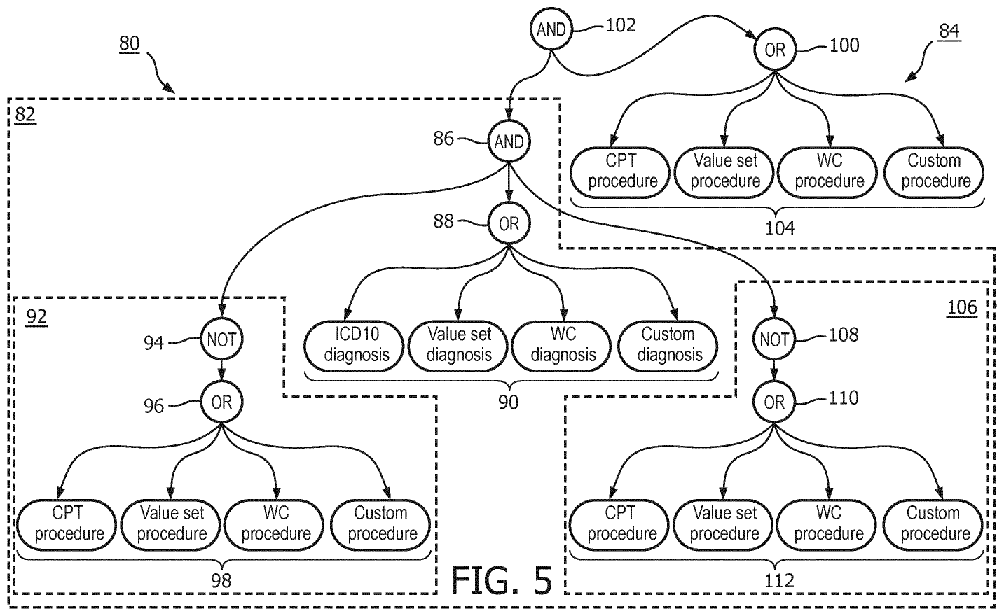

FIG. 5 illustrates an example template tree structure 80 with an example denominator with exclusions and exceptions and numerator item types for an embodiment of an example quality measure analysis system. The template tree structure 80 comprises a denominator portion 82 and a numerator portion 84. The denominator portion 82 comprises in this example a Boolean AND 86 that connects to a Boolean OR 88 that comprises a list of example item types 90, including ICD10 diagnosis codes, value set diagnosis codes, user diagnosis codes, and custom diagnosis codes as explained in association with FIG. 4. As indicated above, additional and/or different item types may be listed. Also, the denominator portion 82 further comprises an exclusion portion 92 comprising a Boolean NOT 94 connected to the Boolean AND 86. The Boolean NOT 94 connects to a Boolean OR 96, which comprises a list of items 98 that comprises, in this example, CPT procedure codes, value set procedure codes, user procedure codes, and custom procedure codes. As is known, denominator exclusions provide a mechanism to exclude patients from the denominator of a performance measure when a therapy or service is not appropriate in instances for which the patient otherwise meets the denominator criteria (e.g., a patient with bilateral lower extremity amputation is excluded from a measure of a diabetic foot exam). The denominator portion 82 further comprises an exceptions portion 106, which comprises a Boolean NOT 108 that connects to a Boolean OR 110, the Boolean NOT 108 also connected to the Boolean AND 86. The Boolean OR 110 comprises a list of items 112, which include in this example CPT procedure codes, value set procedure codes, user procedure codes, and custom procedure codes. The denominator exception comprises an allowable reason for nonperformance of a quality measure for patients that meet the denominator criteria and do not meet the numerator criteria. Denominator exceptions comprise a valid reason for patients whom are included in the denominator population but for whom a process or outcome of care does not occur. Exceptions allow for clinical judgment and fall into three general categories: medical reasons, patient's reasons, and systems reasons. The numerator portion 84 comprises a Boolean OR 100 connected to a root Boolean AND 102 (the latter connecting to the Boolean AND 86 of the denominator portion 82), the Boolean OR 100 comprising a list of items 104 comprising, in this example, CPT procedure codes, value set procedure codes, user procedure codes, and custom procedure codes. Note that the configuration of the template tree structure 80 is one example, and that other configurations may include fewer or additional nodes. Also, it is noted that the list of items are illustrative of one example, and that the items 90, 98, 104, 112 may comprise fewer, additional, and/or different item types. Further, as indicated above, a listing of item types is not limited to an expansion from an OR Boolean node (e.g., may be expanded from a Boolean AND).

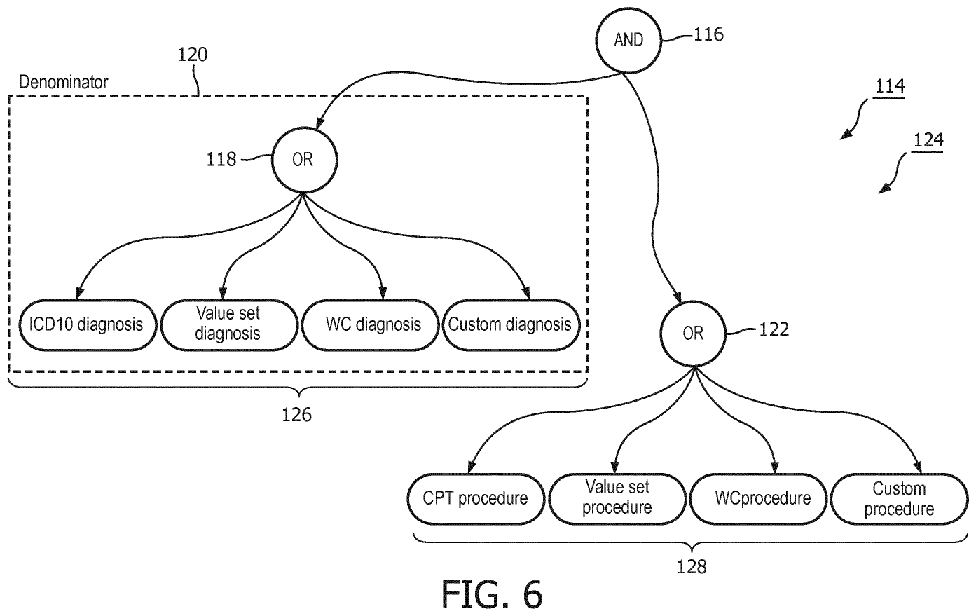

FIG. 6 illustrates an example template tree structure 114 with example denominator and numerator item types, the denominator without exclusions and exceptions for an embodiment of an example quality measure analysis system. Note that not all quality measures have exclusions and/or exceptions. The template tree structure 114 comprises a root Boolean AND 116 that connects to Boolean OR 118 of a denominator portion 120 and to a Boolean OR 122 of a numerator portion 124. The list of example items 126 of the Boolean OR 118 comprises ICD10 diagnostic codes, value set diagnostic codes, user diagnostic codes, and custom diagnosis codes. The list of example items 128 of the Boolean OR 122 comprises CPT procedure codes, value set procedure codes, user procedure codes, and custom procedure codes. Note that additional, fewer, and or/different item types may be included for the list of items 126 and/or 128 in some embodiments, and that those depicted in FIG. 6 are illustrative of one example.

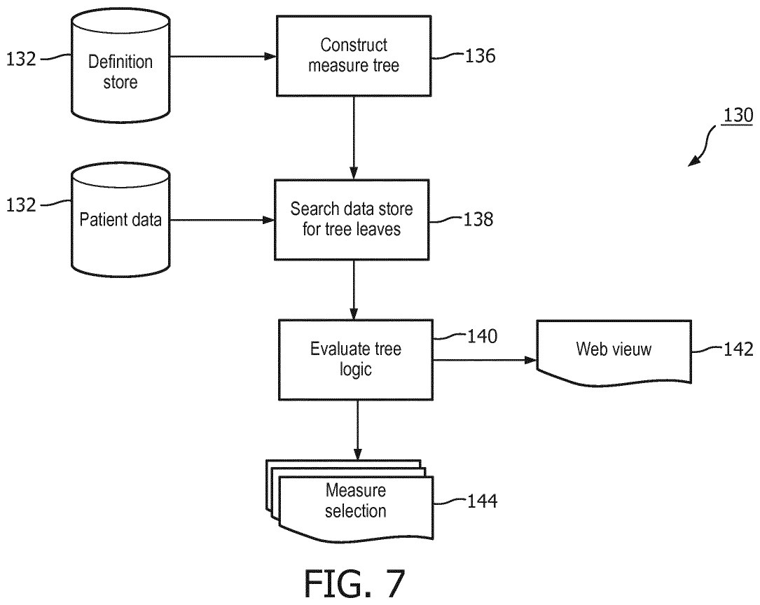

Attention is now directed to FIG. 7, which is a flow diagram that illustrates an example process 130 for building and presenting a tree structure for an embodiment of an example quality measure analysis system. The flow diagram illustrates several components of the process 130, including a definitions store 132, a patients data store 134, logical blocks comprising a construct measure tree block 136, a search data store for tree leaves block 138, an evaluate tree logic block 140, a web view block 142, and a measure selection block 144. The definitions store 132 and patients data store 134 were described in association with FIG. 1B. The construction of the measure tree (136) is based on the inputted data from the definitions store 132, and prepares the tree structure(s), such as the tree structures 36 and 56 shown in FIGS. 2-3. The constructed tree structures omit any patient or record counts. The construction of the measure tree (136) creates the tree structures based on the given quality measure using, for instance, a rules-based scheme. After the construction of the measure tree (136), the patient data store 134 is searched for tree leaves (138), and the tree logic is evaluated (140). The tree leaves comprise nodes that terminate the tree, and exclude the root node. The evaluate tree logic 140 comprises Boolean logic used to evaluate non-leaf nodes, though in some embodiments, may extend beyond simple Boolean operations. In effect, under the processes 138 and 140, the very bottom level of the tree is filled with all of the patient and record counts, and the Boolean logic (using ANDs and ORs) is implemented up the tree, grouping patients and records, until the root level is reached. The web view (142) is presented, in effect presenting the visualization (the completed, tree structures). In one embodiment, the web view (142) is based on a standard REST API and a web-front end. In one embodiment, the web graphical user interface may be located inside a specified virtual private network (VPN). Then, measure selection (144) is implemented (e.g., selection for reporting). Note that selection in the GUI 34, for instance, refers to selection for what to display. Using a batch process that bypasses the GUI 34 gets results for all measures in a much smaller time window, which allows users to know their best performing measures to report to CMS. In one embodiment, measure selection (144) may be performed naively with only the root node, wherein a user with experience and knowledge of data and practices of a customer may briefly review the visualization for measures that are unexpectedly low for opportunities to quickly improve the score. Using PQRS as an example, there are over 200 quality measures that are commonly used, and those quality measures are run through the logic of the measure quality analysis system resulting in a quality measure based on various inputted parameters (e.g., from the fields 42, FIGS. 2-3) along with the quantity of patients eligible for the given condition the quality measure seeks to address. In CMS-based incentive programs, providers need to select up to 9 of the total quality measures (and at least three to avoid penalties). Certain embodiments of a quality measure analysis system run all the quality measures and determine a performance rate (e.g., based on well-documented CMS scoring) for all of these quality measures, and select the top 9 quality measures based on the performance rate. This obviates a manually-intensive, trial and error approach whereby the customer merely selects what they believe to be the best measures, and wait for the results of those selections (e.g., inventive payback). The top level view (root AND) is the result of the measures run all at once, yet the visualization of the additional levels of the tree structures provide a basis for further identification of anomalies and further assists the user in advising the customers.

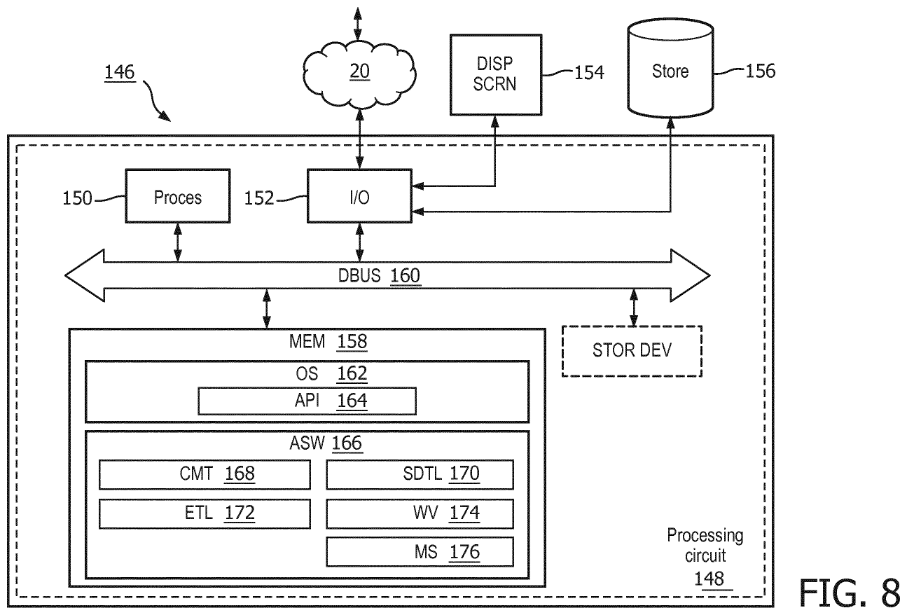

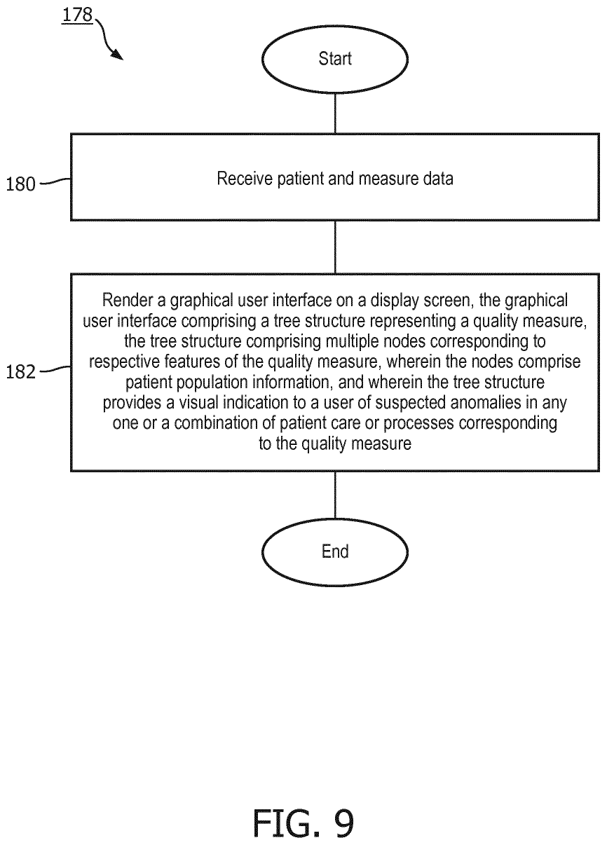

Referring now to FIG. 8, shown is an example computing device 146 that may be used in some embodiments to perform the logic portions of the process 130 depicted in FIG. 7. The computing device 146 may be embodied as an application server device (e.g., server device 12, FIG. 1A) or a client device (e.g., client device 16 of FIG. 1A). One having ordinary skill in the art should appreciate in the context of the present disclosure that the example computing device 146 is merely illustrative of one embodiment, and that some embodiments of computing devices may comprise fewer or additional components, and/or some of the functionality associated with the various components depicted in FIG. 8 may be combined, or further distributed among additional modules or devices, in some embodiments. The computing device 146 is depicted in this example as a computer system. It should be appreciated that certain well-known components of computer systems are omitted here to avoid obfuscating relevant features of the computing device 146. In one embodiment, the computing device 146 comprises a processing circuit 148 that comprises hardware, software, or a combination of hardware and software. In some embodiments, the processing circuit 148 may comprise a greater number, or fewer, components than those depicted in FIG. 8. In one embodiment, the processing circuit 148 comprises one or more processors, such as processor 150 (PROCES 150), input/output (I/O) interface(s) 152 (I/O 152), which in one embodiment is coupled to a display screen 154 (DISP SCRN 154) and one or more data storage devices, including data storage device 156 (STORE 156), and other user interfaces (e.g., keyboard, mouse, microphone, etc.), and memory 158 (MEM 158), all coupled to one or more data busses, including data bus 160 (DBUS 160). In some embodiments, the display screen 154, data storage device 156, and/or other user interfaces may be coupled directly to the data bus 160, or coupled to the computing device 146 over the network 20. The memory 158 may include any one or a combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.). The memory 158 may store a native operating system, one or more native applications, emulation systems, or emulated applications for any of a variety of operating systems and/or emulated hardware platforms, emulated operating systems, etc. In some embodiments, a separate data storage device may be coupled as a network-connected device (or devices) via the I/O interfaces 152 and the network 20. The data storage device 156 may be embodied as persistent memory (e.g., optical, magnetic, and/or semiconductor memory and associated drives) to store patient data (e.g., including cares, medicine, labs, vitals, or collectively, items, diagnosis codes, HCC codes, patient identifiers (IDs), demographics, etc.) and quality measures. In other words, the data storage device 156 may comprise the combined data storage capabilities of the patients data store 134 and the definitions store 132. In some embodiments, the data storage for the patients data store 134 and the definitions store 132 may be separate. In one embodiment, the data storage device 156 may operate according to SQL (e.g., MySQL) processing, though in some embodiments, the processing of the data storage device 156 may be based on non-SQL processing. SQL is an open-source relational database management system that can be run as a virtual machine image or service on a cloud computing platform, including Amazon EC2, among other third party or proprietary platforms. Note that the use of SQL is one example embodiment, and that other relational database management system, including non-SQL data storage processing may be employed in some embodiments. Further, though a single storage device 156 is illustrated, in some embodiments as indicated above, multiple of such data storage devices 156 (or other configurations) may be used. The data may be configured according to a data base structure, though other data structures may be used.

In the embodiment depicted in FIG. 8, the memory 158 comprises an operating system 162 (OS), which includes an application programming interface 164 (API 164). In one embodiment, the API 164 may enable the importation of patient data from another device (e.g., server device) to the storage device 156 and/or the presentation of the web view. The memory 158 further comprises application software 166 (ASW), which in one embodiment includes a construct measure tree (CMT) module 168, a search data store for tree leaves (SDTL) module 170, an evaluate tree logic (ETL) module 172, a web view (WV) module 174, and a measure selection (MS) module 176. These modules 168-176 correspond to the process logic modules 136-144 described in association with the process 130 of FIG. 7, and hence description of the same is omitted here for brevity. In some embodiments, additional or fewer (e.g., combined functionality) modules to provide similar functionality may be used, either co-located in memory 158 (or storage device (STOR DEV)) or distributed among additional devices. In one embodiment, the functionality of the application software 166 may be distributed among plural devices. It is assumed for purposes of illustration that the processing functionality of the application software 166 resides entirely at the computing device 146, with the understanding that the various functional modules of the application software 166 may reside in additional devices. In the depicted embodiment, the web view module 174 provides a web-based user interface, but in some embodiments, the web-view modules 174 may use the API 164 to communicate the trees to another device that operates in, for instance, a server-client relationship with client devices to present the trees at a client device.

Execution of the application software 166 may be implemented by the processor 150 under the management and/or control of the operating system 162. The processor 150 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing device 146.

The I/O interfaces 152 comprise hardware and/or software to provide one or more interfaces to the network(s) 20, as well as to other devices such as the display screen 154, the data storage device 156, and other user interfaces. In other words, the I/O interfaces 152 may comprise any number of interfaces for the input and output of signals (e.g., analog or digital data) for conveyance of information (e.g., data) over various networks and according to various protocols and/or standards. The user interfaces may include a keyboard, mouse, microphone, immersive head set, etc., which enable input and/or output by an administrator or other user.

When certain embodiments of the computing device 146 are implemented at least in part with software (including firmware), as depicted in FIG. 8, it should be noted that the software (e.g., including the application software 166 and its component modules 168-176) can be stored on a variety of non-transitory computer-readable medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing device 146 are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

Having described various embodiments of a quality measure analysis system, it should be appreciated that one embodiment of a quality measure analysis method, denoted as method 178, comprises receiving patient and measure data (180); and rendering a graphical user interface on a display screen, the graphical user interface comprising a tree structure representing a quality measure, the tree structure comprising multiple nodes corresponding to respective features of the quality measure, wherein the nodes comprise patient population information, and wherein the tree structure provides a visual indication to a user of suspected anomalies in any one or a combination of patient care or processes corresponding to the quality measure (182).

Any process descriptions or blocks in the flow diagram illustrated in conjunction with the present description should be understood as representing data, modules, segments, or portions of code which include one or more executable instructions for implementing specific logical functions or steps in the process, and alternate implementations are included within the scope of an embodiment of the present invention in which functions may be executed substantially concurrently and/or in a different order, and/or additional logical functions or steps may be added, depending on the functionality involved, as would be understood by those reasonably skilled in the art of the present invention.

In one embodiment, a quality measure analysis system claim is presented the system comprising: a computing device; and a display screen, wherein the computing device is configured to render a graphical user interface on the display screen, the graphical user interface comprising a tree structure representing a quality measure, the tree structure comprising multiple nodes corresponding to respective features of the quality measure, wherein the nodes comprise patient population information, and wherein the tree structure provides a visual indication to a user of suspected anomalies in any one or a combination of patient care or processes corresponding to the quality measure.

In one embodiment, a quality measure analysis system of the prior claim is presented, wherein one of the multiple nodes comprises a root level, wherein the root level comprises a patient count and a record count for a first condition, the root level comprising a first level, the root level comprising a Boolean AND, a Boolean OR, a Boolean NOT, or a single code.

In one embodiment, a quality measure analysis system of any one of the preceding system claims is presented, wherein a threshold difference between a patient count at the first level and a total patient population provides an indication to the user of a suspected anomaly.

In one embodiment, a quality measure analysis system of any one of the preceding system claims is presented, wherein the Boolean OR or the Boolean AND comprises an ordered list of items corresponding to the first condition, the items comprising any one or a combination of a current procedural terminology code procedure, a value set procedure, an agent procedure, a custom procedure, analytic tests, demographics, tags, industry standard diagnosis codes, value set diagnosis codes, agent diagnosis codes, or custom diagnosis codes.

In one embodiment, a quality measure analysis system of any one of the preceding system claims is presented, wherein the multiple nodes comprise one or more additional levels connected to the root level, each of the one or more additional levels comprising a Boolean AND, a Boolean OR, a Boolean NOT, or a single code.

In one embodiment, a quality measure analysis system of any one of the preceding system claims is presented, wherein the tree structure comprises a numerator component and a denominator component, the numerator component and the denominator component corresponding to respective features of the quality measure, the numerator component and the denominator component comprising one or more levels of the multiple nodes.

In one embodiment, a quality measure analysis system of any one of the preceding system claims is presented, wherein a threshold difference between the patient count of the root level and a patient count corresponding to at least a node of the numerator provides an indication to the user of a suspected anomaly.

In one embodiment, a quality measure analysis system of any one of the preceding system claims is presented, wherein the quality measure features corresponding to the denominator are adjusted for the denominator for one or a combination of exclusions or exceptions, each comprising zero or more items.

In one embodiment, a quality measure analysis system of any one of the preceding system claims is presented, wherein the graphical user interface enables a user to toggle between a view of the numerator component and a view of the denominator component.

In one embodiment, a quality measure analysis system of any one of the preceding system claims is presented, wherein at least one of the multiple nodes comprises a Boolean OR or a Boolean AND, the at least one of the multiple nodes comprising an ordered list of items, each of the items comprising a patient count and a record count for a first condition, the items comprising any one or a combination of current procedural terminology code procedure, a value set procedure, an agent procedure, a custom procedure, analytic tests, demographics, tags, industry standard diagnosis codes, value set diagnosis codes, agent diagnosis codes, or custom diagnosis codes.

In one embodiment, a quality measure analysis system of any one of the preceding system claims is presented, wherein a threshold difference between the record count and the patient count within at least one of the items provides an indication of a suspected anomaly.

In one embodiment, a quality measure analysis system of any one of the preceding system claims is presented, wherein a relative broadness of the top listed item compared to availability of items corresponding to the first condition provides an indication of a suspected anomaly.

In one embodiment, a quality measure analysis system of any one of the preceding system claims is presented, wherein the graphical user interface comprises one or more fields to limit the tree structure to one or more parameters.

In one embodiment, a computer-implemented quality measure analysis method claim is presented, the method comprising: receiving patient and measure data; and rendering a graphical user interface on a display screen, the graphical user interface comprising a tree structure representing a quality measure, the tree structure comprising multiple nodes corresponding to respective features of the quality measure, wherein the nodes comprise patient population information, and wherein the tree structure provides a visual indication to a user of suspected anomalies in any one or a combination of patient care or processes corresponding to the quality measure.

In one embodiment, a computer-implemented quality measure analysis method of the prior method claim is presented, wherein rendering comprises rendering one of the multiple nodes as a root level, wherein the root level comprises a patient count and a record count for a first condition, the root level comprising a first level, the root level comprising a Boolean AND, a Boolean OR, a Boolean NOT, or a single code.

In one embodiment, a computer-implemented quality measure analysis method of any one of the preceding method claims is presented, further comprising providing an indication to a user of a suspected anomaly based on a threshold difference between patient count at the first level and total patient population.

In one embodiment, a computer-implemented quality measure analysis method of any one of the preceding method claims is presented, wherein rendering comprises rendering a numerator component and a denominator component, the numerator component and the denominator component corresponding to respective features of the quality measure, the numerator component and the denominator component comprising one or more levels of the multiple nodes, wherein a threshold difference between the patient count of the root level and a patient count corresponding to at least a node of the numerator provides an indication to the user of a suspected anomaly.

In one embodiment, a computer-implemented quality measure analysis method of any one of the preceding method claims is presented, wherein rendering comprises rendering at least one of the multiple nodes as a Boolean OR or a Boolean AND, the at least one of the multiple nodes comprising an ordered list of items, each of the items comprising a patient count and a record count for a first condition, the items comprising any one or a combination of current procedural terminology code procedure, a value set procedure, an agent procedure, a custom procedure, analytic tests, demographics, tags, industry standard diagnosis codes, value set diagnosis codes, agent diagnosis codes, or custom diagnosis codes.

In one embodiment, a computer-implemented quality measure analysis method of any one of the preceding method claims is presented, further comprising providing an indication to a user of a suspected anomaly based on one or any combination of a threshold difference between the record count and the patient count within at least one of the items or a relative broadness of the top listed item compared to availability of items corresponding to the first condition.

In one embodiment, a non-transitory computer readable medium claim is presented, the non-transitory computer readable medium claim encoded with executable instructions that, when executed by one or more processors, causes the one or more processors to: receive patient and measure data; and render a graphical user interface on a display screen, the graphical user interface comprising a tree structure representing a quality measure, the tree structure comprising multiple nodes corresponding to respective features of the quality measure, wherein the nodes comprise patient population information, and wherein the tree structure provides a visual indication to a user of suspected anomalies in any one or a combination of patient care or processes corresponding to the quality measure.

Note that reference to thresholds refers to minimum triggers for certain conditions for actions to commence. The thresholds may be based on historical or experimental data, or based on the expertise of a user and/or context. In some embodiments, the threshold may be established based on a combination of experience and context. For instance, evidence of a suspected anomaly may be dependent on the condition of the patient, such as a scenario where a healthy person is expected to have one wellness visit per year, whereas someone with a chronic illness may be expected to have a schedule of visits.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. For instance, though the discussion focuses on the US-based government-reimbursed healthcare services (e.g., Medicare), it should be appreciated by one having ordinary skill in the art that certain embodiments of a quality measure analysis system may be used in any incentive programs in the US or elsewhere based on a measure that may be defined and a coding system. Further, though the quality measure builder examples depicted in FIGS. 2-3 shows numerator and denominator values, the values provides for saved searches or alerts, for instance, provide a single value. An alert, for instance, may be alerting the user (or customer) of another visit to a service period within a prescribed period by the patient after a discharge from the ER (a metric that is to be reduced, since another visit may reflect a re-occurrence of the condition that lead to the prior ER visit). Another variation of an alert is the monitoring of an encouraged follow-up visit by a patient within a prescribed period of a discharge, such as to ensure medicine compliance, to avoid downward adjustments to performance scores. As another example of variations, certain embodiments of the quality measure analysis system may be used via command-line via tools like curl to make the REST API. The command-line can be used to batch multiple REST API calls together. Further, though a user is described in the example above for which the graphical user interfaces 34 are presented for interpretation and analysis, it should be appreciated by one having ordinary skill in the art that the user may be of or associated with a customer or a third party (e.g., consultant). Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

1. A quality measure analysis system, comprising:
a computing device; and
a display screen, wherein the computing device is configured to render a graphical user interface on the display screen, the graphical user interface comprising a tree structure representing a quality measure, the tree structure comprising multiple nodes corresponding to respective features of the quality measure, wherein the nodes comprise patient population information, and wherein the tree structure provides a visual indication to a user of suspected anomalies in any one or a combination of patient care or processes corresponding to the quality measure to trigger additional investigation.

2. The system of claim 1, wherein one of the multiple nodes comprises a root level, wherein the root level comprises a patient count and a record count for a first condition, the root level comprising a first level, the root level comprising a Boolean AND, a Boolean NOT, a Boolean OR, or a single code.

3. The system of claim 2, wherein a threshold difference between a patient count at the first level and a total patient population provides an indication to the user of a suspected anomaly.

4. The system of claim 2, wherein the Boolean OR or the Boolean AND comprises an ordered list of items corresponding to the first condition, the items comprising any one or a combination of a current procedural terminology code procedure, a value set procedure, an agent procedure, a custom procedure, analytic tests, demographics, tags, industry standard diagnosis codes, value set diagnosis codes, agent diagnosis codes, or custom diagnosis codes.

5. The system of claim 2, wherein the multiple nodes comprise one or more additional levels connected to the root level, each of the one or more additional levels comprising a Boolean AND, a Boolean OR, a Boolean NOT, or a single code.

6. The system of claim 1, wherein the tree structure comprises a numerator component and a denominator component, the numerator component and the denominator component corresponding to respective features of the quality measure, the numerator component and the denominator component comprising one or more levels of the multiple nodes.

7. The system of claim 6, wherein a threshold difference between the patient count of the root level and a patient count corresponding to at least a node of the numerator provides an indication to the user of a suspected anomaly.

8. The system of claim 6, wherein the quality measure features corresponding to the denominator are adjusted for the denominator for one or a combination of exclusions or exceptions, each comprising zero or more items.

9. The system of claim 6, wherein the graphical user interface enables a user to toggle between a view of the numerator component and a view of the denominator component.

10. The system of claim 1, wherein at least one of the multiple nodes comprises a Boolean OR or a Boolean AND, the at least one of the multiple nodes comprising an ordered list of items, each of the items comprising a patient count and a record count for a first condition, the items comprising any one or a combination of current procedural terminology code procedure, a value set procedure, an agent procedure, a custom procedure, analytic tests, demographics, tags, industry standard diagnosis codes, value set diagnosis codes, agent diagnosis codes, or custom diagnosis codes.

11. The system of claim 10, wherein a threshold difference between the record count and the patient count within at least one of the items provides an indication of a suspected anomaly.

12. The system of claim 10, wherein a relative broadness of the top listed item compared to availability of items corresponding to the first condition provides an indication of a suspected anomaly.

13. The system of claim 1, wherein the graphical user interface comprises one or more fields to limit the tree structure to one or more parameters.

14. A computer-implemented quality measure analysis method, comprising:
receiving patient and measure data; and
rendering a graphical user interface on a display screen, the graphical user interface comprising a tree structure representing a quality measure, the tree structure comprising multiple nodes corresponding to respective features of the quality measure, wherein the nodes comprise patient population information, and wherein the tree structure provides a visual indication to a user of suspected anomalies in any one or a combination of patient care or processes corresponding to the quality measure trigger additional investigation.

15. The method of claim 14, wherein rendering comprises rendering one of the multiple nodes as a root level, wherein the root level comprises a patient count and a record count for a first condition, the root level comprising a first level, the root level comprising a Boolean AND, a Boolean OR, a Boolean NOT, or a single code.

16. The method of claim 15, further comprising providing an indication to a user of a suspected anomaly based on a threshold difference between patient count at the first level and total patient population.

17. The method of claim 15, wherein rendering comprises rendering a numerator component and a denominator component, the numerator component and the denominator component corresponding to respective features of the quality measure, the numerator component and the denominator component comprising one or more levels of the multiple nodes, wherein a threshold difference between the patient count of the root level and a patient count corresponding to at least a node of the numerator provides an indication to the user of a suspected anomaly.

18. The method of claim 14, wherein rendering comprises rendering at least one of the multiple nodes as a Boolean OR or a Boolean AND, the at least one of the multiple nodes comprising an ordered list of items, each of the items comprising a patient count and a record count for a first condition, the items comprising any one or a combination of current procedural terminology code procedure, a value set procedure, an agent procedure, a custom procedure, analytic tests, demographics, tags, industry standard diagnosis codes, value set diagnosis codes, agent diagnosis codes, or custom diagnosis codes.

19. The method of claim 18, further comprising providing an indication to a user of a suspected anomaly based on one or any combination of a threshold difference between the record count and the patient count within at least one of the items or a relative broadness of the top listed item compared to availability of items corresponding to the first condition.

20. A non-transitory computer readable medium encoded with executable instructions that, when executed by one or more processors, causes the one or more processors to:
receive patient and measure data; and
render a graphical user interface on a display screen, the graphical user interface comprising a tree structure representing a quality measure, the tree structure comprising multiple nodes corresponding to respective features of the quality measure, wherein the nodes comprise patient population information, and wherein the tree structure provides a visual indication to a user of suspected anomalies in any one or a combination of patient care or processes corresponding to the quality measure to trigger additional investigation.
